(19) United States
(12) Patent Application Publication
(10) Pub. No.: US 2024/0382137 A1
(43) Pub. Date: Nov. 21, 2024

(54) PROCESS FOR MANUFACTURING A GARMENT FOR THE ACQUISITION OF ELECTROMYOGRAPHIC SIGNALS

(71) Applicant: ETA BIOENGINEERING S.R.L., Castellammare eli Stabia (NA) (IT)

(72) Inventors: Teodorico CAPORASO, Benevento (IT); Stanislao GRAZIOSO, Castellammare eli Stabia (NA) (IT); Angela PALOMBA, Gragnano (NA) (IT); Dario PANARIELLO, Colli del Tronto (AP) (IT); Anna GRAZIOSO, Castellammare eli Stabia (NA) (IT); Michele CAPORASO, Benevento (IT); Giuseppe DI GIRONIMO, Napoli (IT); Antonio LANZOTTI, Napoli (IT)

(21) Appl. No.: 18/692,021

(22) PCT Filed: Sep. 14, 2022
(86) PCT No.: PCT/EP2022/075578

§ 371 Date: Mar. 14, 2024

(30) Foreign Application Priority Data
Sep. 15, 2021 (IT) 102021000023753

(51) Int. Cl. A61B 5/389; A61B 5/256

(52) U.S. Cl. CPC A61B 5/389; A61B 5/256; A61B 2503/10; A61B 2562/12

(57) ABSTRACT

A process for manufacturing a garment for acquiring electromyographic signals is described comprising the step of providing a garment having dimensions consistent with a three-dimensional digital model, previously provided, of a wearer's anatomical shapes, wherein said at least one electromyography (EMG) electrode device, optionally a textile electrodes or printed electrodes, is positioned on a garment as a function of positioning coordinates previously calculated from said three-dimensional digital model.

### FIELD OF APPLICATION

The present invention relates to a process for manufacturing a garment, in particular a pair of shorts, a shirt, a sleeve, a pair of socks, designed to acquire electromyographic signals.

Specifically, the garment thus manufactured comprises a plurality of electromyography (EMG) sensors.

The garment thus manufactured is useful to quantify the muscle activity of the wearer's muscles in proximity of which such electromyography sensors are located and is particularly useful for sports and clinical applications.

### PRIOR ART

By using garments comprising electromyography (EMG) sensors, it is possible to obtain specific information on athletes during training and/or competition activities.

The information thus gathered may then be used to monitor and build progressions in training sessions.

Furthermore, the information acquired may enable the coach to assess the potential risk of de-training during the season (often underlying possible injuries).

In addition, in the step of recovering from an injury, an electromyography garment provides accurate measures of what the athlete's actual state is, in order to properly manage the so-called “return to play”.

The use of the electromyography technique by means of surface electrodes (sEMG) in garments, in particular garments that can be used while performing sports activities, is well known.

In this framework, the use of the electromyography technique allows: (i) high sampling rates and (ii) higher precision in positioning them on single muscles.

By contrast, surface electrode electromyography (sEMG) has the disadvantage that it is not suitable for dynamic activities due to the loss of adhesion during activity, e.g. sports activity. In addition, multiple acquisition modules are often required with associated weight issues.

These limitations have therefore shifted general interest in recent years towards the use of electromyographic evaluation by means of textile electrodes in the sports field.

An electromyography device by means of electromyography (EMG) electrodes is described in US 2015/0148619 A1 and comprises a garment configured to be worn by the user and comprising a set of biometric sensors coupled to the garment and configured to communicate with the measuring module, and the measuring module itself to receive and transmit biometric signals indicative of the user's muscle activity.

The biometric sensors include electromyography (EMG) electrodes configured to acquire potential signals resulting from the user's muscle activity, positioned in the desired positions when the garment is worn by the user.

US 2020/0022651 A1 relates to a pair of sensorised shorts for monitoring training by measuring the activity of the body's muscles exploiting electromyography, wherein the electrical signals caused by active muscles are measured by means of so-called textile electrodes, having a diameter lower than 1 cm, positioned on the surface of the skin. The conductors are made of conductive textile material.

For better signal acquisition by the electrodes, it is recommended to remove hair and keep the skin surface at the electrode points as clean as possible, as well as to use conductive gel between the electrodes and the skin.

A limitation connected to the use of textile electrodes is therefore the need to maintain sufficient contact between the EMG textile electrodes and the muscle of interest. If contact between the EMG textile electrodes and the muscle of interest is not sufficient, it is not possible to gather meaningful data.

The solution to this drawback is to adequately lubricate the areas where the electrodes come into contact with the muscles, in particular the leg muscles, especially for users who do not produce enough sweat. In the latter case, in fact, only by applying lubricant it is possible to ensure that the shorts fit the wearer well and produce a reliable EMG signal.

The current limitations of electromyography by means of textile electrodes are in any case the following ones:

(i) impossibility in evaluating the muscle activity of single muscles (the estimation of the activity of specific muscle regions, e.g. the quadriceps, is generally preferred);

(ii) variability of the position of single muscles in relation to the location of the muscle region (due to different anthropometric characteristics).

US 2020/261023 A1 discloses an athlete garment and a process for providing it, said athlete garment comprising a plurality of sensors, in particular EMG sensors, wherein the selection of sensor placement sites is made to match locations of features of musculoskeletal structure of athlete participant, such as innervation zones.

Such process comprises a calibration procedure during which grids of sEMG sensors are attached, in a calibration garment, to regions of “likely innervation zones”; then, data detected from sEMG sensors are collected and readjustment is made for the locations of the sEMG sensors.

Hence, in order to obtain a functional athlete garment comprising a plurality of sensors, the athlete must wear a calibration garment and participate to a calibration procedure.

The need to make available an electromyography garment that is free of the drawbacks mentioned above with reference to the prior art is particularly perceived.

The technical problem underlying the present invention is therefore to make available an electromyographic garment which allows to quantify precisely and in a personalised manner, in terms of electrical potential difference, the muscle activity while performing movements, and, in particular, which allows to evaluate the muscle activity of single muscles belonging to different muscle regions, with a limited cross talk phenomena with adjacent muscles and, more particularly, the muscle activity of single muscles, belonging to different muscle regions, with a limited cross talk phenomena with adjacent muscles, during sports, work and clinical movements.

### SUMMARY OF THE INVENTION

Such technical problem is solved, according to the present invention, by a process for manufacturing a garment for acquiring electromyographic signals comprising the following steps:

a) providing a three-dimensional digital model of a wearer's anatomical shapes;

b) automatically deriving from such a three-dimensional digital model, anatomical landmarks of the wearer's body parts;

c) automatically calculating from such anatomical landmarks, positioning coordinates of at least one electromyography (EMG) electrode device on such three-dimensional digital model;

d) providing a garment having dimensions consistent with such a three-dimensional digital model of a wearer's anatomical shapes, thus provided during step a), such at least one electromyography (EMG) electrode being positioned on such a garment according to the positioning coordinates calculated during step c).

Preferably, said electromyography (EMG) electrode device is a textile electrode device or a printed electrode device, more preferably a textile electrode device.

Advantageously, the process according to the present invention allows to make available an electromyographic garment capable of quantifying in a precise and personalised manner, in terms of electrical potential difference, the muscle activity while performing movement activities and, in particular, allows to evaluate the muscle activity of single muscles with a limited cross talk phenomena with adjacent muscles.

In particular, in case of a plurality of electromyography (EMG) electrode devices, the process according to the present invention allows to make available an electromyographic garment adapted to quantify, in precise and personalised manner, the muscle activity of single muscles belonging to different muscle regions during human movements, wherein each electromyography (EMG) electrode device is configured to quantify the muscle activity of a single muscle of a specific muscle region, without a limited cross talk phenomena with adjacent muscles.

Thus, thanks to a specific design methodology, which is in particular carried out in steps a), b) and c), the process according to the invention allows to solve the aforementioned technical problem.

Preferably, the step a) of providing a three-dimensional digital model comprises the following sub-steps:
scanning the wearer's anatomical shapes, thereby acquiring information about such anatomical shapes;
reconstructing a three-dimensional digital model of the wearer's anatomical shapes from the information thus acquired.

More preferably, in the sub-step of reconstructing a three-dimensional digital model, such three-dimensional digital model is reconstructed by photogrammetry.

Even more preferably, in the sub-step of scanning human anatomical shapes, information about the wearer's anatomical shapes is associated with images of human anatomical shapes acquired by means of a scanner comprising cameras capable of acquiring images instantaneously and simultaneously.

In particular, the step of scanning human anatomical shapes is carried out according to ISO 20685-1:2018.

More in particular, during the sub-step of scanning human anatomical shapes, the wearer, i.e. the subject to be photographed, is in an upright position with the head looking forward, the feet are parallel to the shoulders, the shoulder joints are abducted at approximately 20° to the sides of the torso, the elbows are slightly flexed and the palms of the hands are facing each other.

In accordance with the present invention, the term “electromyography electrode device” means a sensor comprising a plurality of electromyography (EMG) electrodes, in particular at least two electromyography (EMG) electrodes with opposite polarity.

In particular, according to step c) of automatically calculating coordinates for positioning the at least one electromyography (EMG) electrode device and to step d) of providing a garment, such at least one electromyography (EMG) electrode device comprises a first electromyography (EMG) electrode and a second electromyography (EMG) electrode, wherein the first electromyography (EMG) electrode and the second electromyography (EMG) electrode are positioned on the garment so that they both lie along the same muscle.

More in particular, according to this last embodiment, during step c) of calculating the positioning co-ordinates of at least one electromyography (EMG) electrode device, the positioning coordinates of such first electromyography (EMG) electrode and such electromyography (EMG) second electrode are calculated.

Even more in particular, during the subsequent step d) of providing a garment, the first electromyography (EMG) electrode and the second electromyography (EMG) electrode are positioned on that garment as a function of the positioning coordinates thus calculated during step c).

Advantageously, once the wearer has put on such garment, such first electromyography (EMG) electrode and second electromyography (EMG) electrode will be in contact with a specific muscle, in particular the same muscle.

Thanks to such positioning, it will thus be possible to quantify, in terms of electrical potential difference, specifically between the first electromyography (EMG) electrode and the second electromyography (EMG) electrode, the muscle activity of the same muscle, for example while performing sports activity.

According to the present process, such a garment may be a pair of shorts, a shirt, a sleeve or a pair of socks.

For instance, when such garment is a pair of shorts, such first electromyography (EMG) electrode and such second electromyography (EMG) electrode may be positioned on the garment so that they both lie on the rectus femoris muscle, either of the right leg or left leg.

Preferably, such at least one electromyography (EMG) electrode device is a textile electrode device.

More preferably, in such at least one electromyography (EMG) electrode device such first electromyography (EMG) electrode and such second electromyography (EMG) electrode are textile electrodes.

Preferably, the step b) of automatically deriving, from such a three-dimensional digital model, anatomical landmarks of the wearer's body parts can be carried out according to the process of extracting an anatomical landmark comprising the following sub-steps:
(i) defining the area of interest on the body surface;
(ii) analysing the local curve in the selected area on that three-dimensional digital model;
(iii) extracting the landmark from the local curve analysis.

In accordance with the present invention, the term “electromyography textile electrode” means an electrode configured to measure the electrical activity generated during muscle contraction, which is made using knitting techniques and with conductive material, preferably a conductive metal, more preferably silver.

In particular, the step c) of automatically calculating coordinates for positioning at least one electromyography (EMG) electrode device on such three-dimensional digital model comprises the sub-step of identifying a curve joining a first anatomical landmark and a second anatomical landmark, wherein the coordinates for positioning such at least one electromyography (EMG) electrode device identify a pair of positioning points belonging to such curve, in particular a first positioning point for such first electromyography (EMG) electrode and a second positioning point for such second electromyography (EMG) electrode.

Specifically, the aforementioned curve joining a first anatomical landmark and a second anatomical landmark is the shortest curved line joining the first anatomical landmark and the second anatomical landmark following the surface of such three-dimensional digital model.

More in particular, as will be seen below in relation to the detailed description, such a sub-step of identifying a curve joining a first anatomical landmark and a second anatomical landmark may include the following further sub-steps:
identifying a curve joining such first anatomical landmark and such second anatomical landmark, wherein that curve is the shortest curved line joining the first anatomical landmark and the second anatomical landmark following the surface of such three-dimensional digital model;
identifying a construction point on such curve, such construction point being in a position between such first anatomical landmark and such second anatomical landmark;
identifying the first positioning point for the first electromyography (EMG) electrode and the second positioning point for the second electromyography (EMG) electrode, on that curve, wherein the first positioning point for the first electromyography (EMG) electrode and the second positioning point for the second electromyography (EMG) electrode are symmetrical, along that curve, with respect to such construction point.

Preferably, in said sub-steps of identifying a construction point on said curve, in said position of the construction point the curve has the maximum curvature.

More preferably, such first anatomical landmark and such second anatomical landmark are selected in accordance with the requirements of the SENIAM (Surface ElectroMyoGraphy for the Non-Invasive Assessment of Muscles) guidelines for the muscle under examination (e.g. great trochanter and sacral vertebra for the gluteus maximus muscle).

Accordingly, said construction point is identified in accordance with the requirements of the SENIAM guidelines for the muscle under examination (e.g. great trochanter and sacral vertebra for the gluteus maximus muscle are the anatomical landmarks used to define a curve, according to SENIAM guidelines, it is possible to calculate the positioning coordinates, on said three-dimensional digital model, of at least one electromyography electrode device).

In particular, the step d) of providing a garment may comprise the following sub-steps:
automatically deriving anthropometric measurements of the wearer's body parts from such a three-dimensional digital model;
obtaining, from the anthropometric measures of the wearer's body parts thus derived, the dimensions of the garment;
manufacturing a garment having the dimensions thus obtained, wherein said garment has dimensions consistent with said three-dimensional digital model of a wearer's anatomical shapes, thus provided during step a), said at least one electromyography (EMG) electrode device, preferably a textile electrode device, being positioned on said garment as a function of said positioning coordinates.

Preferably, the sub-step of automatically deriving anthropometric measures from such a three-dimensional digital model involves deriving multiple anthropometric measures. In particular, in the case of a short, the sub-step of automatically deriving anthropometric measures from such a three-dimensional digital model involves deriving at least six anthropometric measures, including the following anatomical measures: waist circumference (i.e. circumference assessed at the height of the anterior iliac spines), the greatest thigh circumference (i.e. the maximum value of the thigh circumference), the smallest thigh circumference (i.e. the minimum value of the thigh circumference) the hip height (i.e. the vertical distance between the iliac spines and the level of the pubic symphysis), the thigh length (i.e. the vertical distance between the level of the pubic symphysis and the lower third of the femur) and the crotch arch length (i.e. the distance between the two thighs assessed at the level of the pubic symphysis, in the standard posture).

More in particular, during the sub-step of obtaining the dimensions of the garment, the dimensions of the garment are calculated taking into account the elasticity of the material to be used for manufacturing the garment.

More preferably, the sub-step of manufacturing the garment is carried out with the aid of electronically controlled circular textile machines.

Advantageously, by using electronically controlled circular knitting machines, the textile products thus obtained are essentially free of additional stitching, as most of the stitching is done automatically by the textile machine and is therefore not detectable on the finished garment.

Preferably, in step d) of providing a garment, such garment is a pair of shorts, a shirt, a sleeve or a pair of socks. More preferably, in step d) of providing a garment, such garment is a pair of shorts.

Equally preferably, in step d) of providing a garment, such garment is made of a stretch fabric.

More preferably, stretch fabric comprises a material selected from the group comprising polyamide, elastane (i.e., spandex), polyester, nylon or any combination thereof.

According to a preferred embodiment of the present process, following the step d) of providing a garment, the following further steps may be carried out:

e) optimising the dimensioning of such first electromyography (EMG) electrode and such second electromyography (EMG) electrode and/or optimising the distance between such first electromyography (EMG) electrode and such second electromyography (EMG) electrode by measuring the electric potential difference between the first electromyography (EMG) electrode and the second electromyography (EMG) electrode, obtaining an optimisation outcome;

f) repositioning such first electromyography (EMG) electrode and such second electromyography (EMG) electrode on that garment according to that optimisation outcome.

In other words, the process according to the present invention not only allows to make available an electromyographic garment capable of quantifying, in a precise and personalised manner, in particular thanks to the above-mentioned sub-steps a) to d), but, thanks to the following sub-steps e) and f), with reference to the embodiments described in detail below, it is possible to optimise the dimensioning and positioning of the electromyography (EMG) electrodes on such garment, in such a way as to further customise the garment manufacturing, making it possible to precisely quantify the muscle activity, in terms of electrical potential difference, while performing sports activities.

In particular, thanks to this process, an electromyographic garment adapted to assess the muscle activity of muscles belonging to different muscle regions during sports movements is made available.

Specifically, it is understood that the optimisation step e) is carried out after the wearer has put the garment on.

Preferably, the optimisation step e) is carried out by measuring the electric potential difference between such first electromyography (EMG) electrode and such second electromyography (EMG) electrode when the wearer performs a specific movement, during which that muscle, on which the first electromyography (EMG) electrode and the second electromyography (EMG) electrode are positioned, is in a contracted state.

More preferably, the optimisation step e) is carried out by measuring the electric potential difference between such first electromyography (EMG) electrode and such second electromyography (EMG) electrode when the wearer performs, as an example, an athletic movement, even more preferably an athletic movement typical of football.

For example, the optimisation step e) may be carried out by measuring the electric potential difference between such first electromyography (EMG) electrode and such second electromyography (EMG) electrode when the wearer performs an athletic movement selected from volley or ball control.

More preferably, the optimisation step e) may be carried out by measuring the electric potential difference between such first electromyography (EMG) electrode and such second electromyography (EMG) electrode by varying the distance between such first electromyography (EMG) electrode and such second electromyography (EMG) electrode on such garment.

Alternatively, the optimisation step e) may be carried out by measuring the electric potential difference between such first electromyography (EMG) electrode and such second electromyography (EMG) electrode by varying the dimensions of such first electromyography (EMG) electrode and/or such second electromyography (EMG) electrode on such garment.

In particular, such first electromyography (EMG) electrode and such second electromyography (EMG) electrode on such garment may be circular electrodes.

More specifically, when such first electromyography (EMG) electrode and such second electromyography (EMG) electrode on that garment are circular electrodes, during such optimisation step e), the diameter of such first electromyography electrode and/or such second electromyography (EMG) electrode on that garment may be varied.

Even more preferably, the optimisation step e) may be carried out by additionally measuring the electric potential difference between such first electromyography (EMG) electrode and such second electromyography (EMG) electrode by varying the position of such first electromyography

(EMG) electrode and such second electromyography (EMG) electrode on the garment in terms of positioning relative to muscles located on the opposite mirror-like side (right or left) of the wearer's body.

Even more preferably, the optimisation step e) may be carried out by additionally measuring the electric potential difference between such first electromyography (EMG) electrode and such second electromyography (EMG) electrode by varying the position of such first electromyography (EMG) electrode and such second electromyography (EMG) electrode on the garment in terms of positioning relative to the variation of the sweating conditions of the wearer.

Even more preferably, the optimisation step e) may be carried out by additionally measuring the electric potential difference between such first electromyography (EMG) electrode and such second electromyography (EMG) electrode by varying the position of such first electromyography (EMG) electrode and such second electromyography (EMG) electrode on the garment in terms of positioning relative to muscles located on the opposite mirror-like side (right or left) of the wearer's body, and

additionally measuring the electric potential difference between such first electromyography (EMG) electrode and such second electromyography (EMG) electrode by varying the position of such first electromyography (EMG) electrode and such second electromyography (EMG) electrode on the garment in terms of positioning relative to the variation of the sweating conditions of the wearer.

Most preferably, the optimisation step e) may be carried out by measuring the electric potential difference between such first electromyography (EMG) electrode and such second electromyography (EMG) electrode according to a factorial plan that is based on the following control factors:

variation of the distance between such first electromyography (EMG) electrode and such second electromyography (EMG) electrode on such garment;

variation of the dimensions of such first electromyography (EMG) electrode and/or such second electromyography (EMG) electrode on such garment;

optionally, variation of the position of such first electromyography (EMG) electrode and such second electromyography (EMG) electrode on the garment in terms of positioning relative to muscles located on the opposite mirror-like side (right or left) of the wearer's body; and,

optionally, variation of the position of such first electromyography (EMG) electrode and such second electromyography (EMG) electrode on the garment in terms of positioning relative to the variation of the sweating conditions of the wearer.

Ultimately, the present process allows to achieve the following advantages:
Customised positioning of textile electrodes;
Possibility of monitoring single muscles and not just entire muscle regions (e.g. it is possible to individually monitor the four single muscles that make up the quadriceps).
Dimensioning of textile electrodes and distance between electrodes through a experimental campaign using the robust design approach;
Robustness of the system to different skin lubrication conditions and variability between dominant and non-dominant limbs;
Minimizing cross talk phenomena between adjacent muscles.

The aforementioned technical problem is also solved by a garment comprising a base structure, at least one electromyography (EMG) electrode device and a control unit, such control unit being connected to such at least one electromyography (EMG) electrode device and being configured to receive a plurality of signals from such at least one electromyography (EMG) electrode device, manufactured by means of the aforementioned process.

Preferably, said electromyography (EMG) electrode device is a textile electrode device.

Preferably, such control unit is configured to process information associated with such plurality of signals from at least one electromyography (EMG) electrode device.

Advantageously, in fact, also thanks to such control unit, it is possible, through the aforementioned garment, to acquire information related to a specific athletic movement, e.g. an athletic movement typical of football, and process it, making it possible to control the performance of such athletic movement by the wearer.

The above-mentioned garment may also comprise a unit for transmitting information, optionally in a wireless mode.

Advantageously, also thanks to such a unit for transmitting information, optionally in a wireless mode, the aforementioned garment allows transmitting such information or the information processing outcome to an element external to the garment according to the present invention.

The characteristics and advantages of the process according to the invention will become clear from the detailed description, made below, and of embodiments thereof given by way of non-limiting example with reference to the attached drawings.

### BRIEF DESCRIPTION OF THE DRAWINGS

FIG. 1 depicts a flowchart showing, in a simplified manner, the steps of an embodiment of the process according to the present invention.

FIG. 2 shows an example of a process for extracting an anatomical landmark from a three-dimensional digital model of a subject.

FIG. 3 shows the two principal curvatures (of a point belonging to a surface of such a three-dimensional digital model, from which the Gauss curvature for the point is calculated.

FIG. 4 shows a graphical construction for identifying the positioning point of a textile electrode on a three-dimensional digital model of a subject; in FIG. 4 it is also shown in detail the identification, starting from point E, of the first positioning point and the second positioning point for a pair of EMG electrodes with a specific diameter and inter-electrode distance.

FIG. 5 shows a pair of shorts manufactured according to an embodiment of the process of the invention according to a front view (A) and according to a rear view (B).

FIG. 6 shows a graphic representation of a wiring diagram of the pair of shorts shown in FIG. 5.

FIG. 7 shows a schematic view of a pair of shorts manufactured according to a different embodiment of the process of the invention, according to a front view.

### DETAILED DESCRIPTION OF PREFERRED EMBODIMENTS

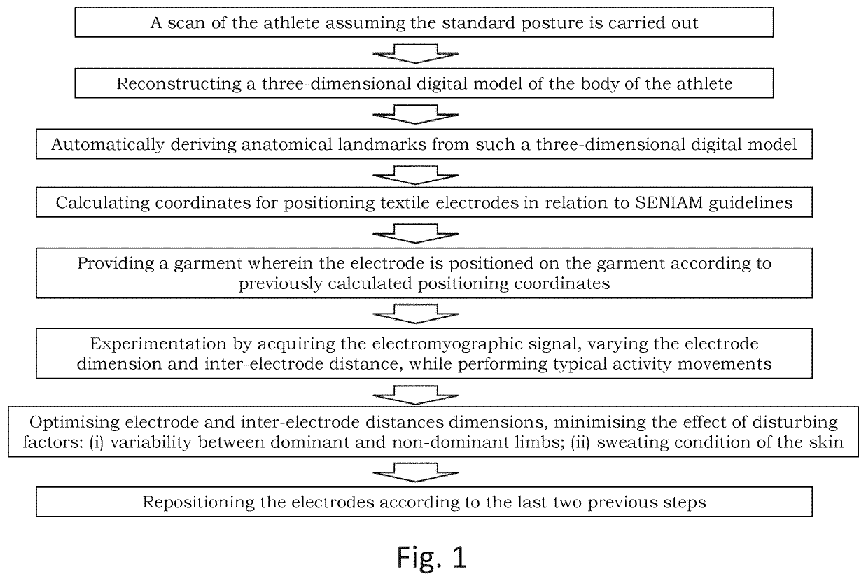

FIG. 1 illustrates a flowchart showing in a simplified manner the following steps and sub-steps of an embodiment of the invention process, wherein the step a) of providing a three-dimensional digital model comprises the following sub-steps:
scanning the wearer's anatomical shapes, acquiring information about such anatomical shapes (first step of FIG. 1);
reconstructing a three-dimensional digital model of the wearer's anatomical shapes from the information thus acquired (second step of FIG. 1).

Thereby, the steps b) and c) follow, i.e.

b) automatically deriving, from such a three-dimensional digital model, anatomical landmarks of the wearer's body parts (third step of FIG. 1); and

c) automatically calculating, from such anatomical landmarks, the positioning coordinates, on such a three-dimensional digital model, of at least one electromyography (EMG) electrode device (fourth step of FIG. 1).

As illustrated in FIG. 1, the step c) of automatically calculating the coordinates for positioning, in such a three-dimensional digital model, at least one electromyography (EMG) electrode device is carried out according to the SENIAM guidelines.

This is followed by the step d) of providing a garment having dimensions consistent with such a three-dimensional digital model of a wearer's anatomical shapes, thus provided during step a), wherein the at least one electromyography (EMG) electrode is positioned on the garment according to the positioning coordinates calculated during step c).

Finally, the following additional steps e) and f) are carried out:

e) optimising the dimensioning of the at least two electromyography (EMG) electrodes mentioned above and/or verifying the distance between such at least two electromyography (EMG) electrodes by measuring the potential difference between such first electromyography (EMG) electrode and such second electromyography (EMG) electrode, obtaining an optimisation outcome;

f) repositioning such first electromyography (EMG) electrode and such second electromyography (EMG) electrode on such garment according to such optimisation outcome (eighth step in FIG. 1);

wherein the optimisation step e) is carried out by measuring the potential difference between such first electromyography (EMG) electrode and such second electromyography (EMG) electrode according to a factorial plan that is based on the following control factors:

variation of the distance between such first electromyography (EMG) electrode and such second electromyography (EMG) electrode on such garment, and

variation of the dimensions of such first electromyography (EMG) electrode and/or such second electromyography (EMG) electrode on such garment (sixth step in FIG. 1);

variation of the position of such first electromyography (EMG) electrode and such second electromyography (EMG) electrode on the garment in terms of positioning relative to groups or muscles located on the opposite mirror-like side (right or left) of the wearer's body, and

variation of the position of such first electromyography (EMG) electrode and such second electromyography (EMG) electrode on the garment in terms of positioning relative to the variation in the sweating conditions of the wearer (seventh step in FIG. 1).

In particular, step b) is preferably performed using the analysis of the curvatures identified on a wearer's body surface, such as an athlete, and, from the scan of the athlete's body, the coordinates of the main anatomical landmarks are identified.

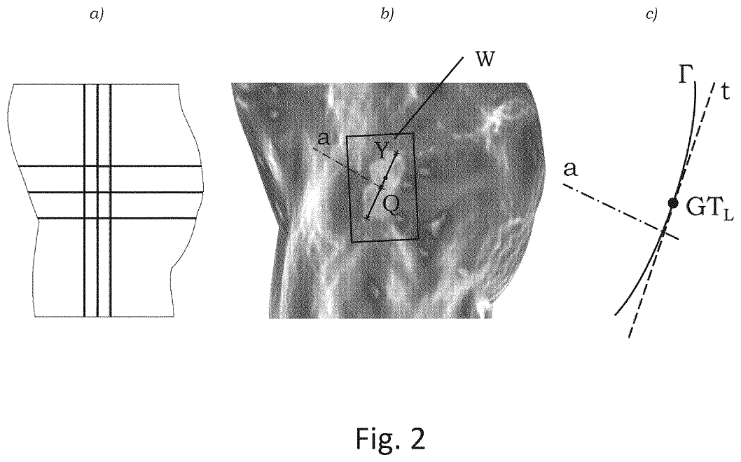

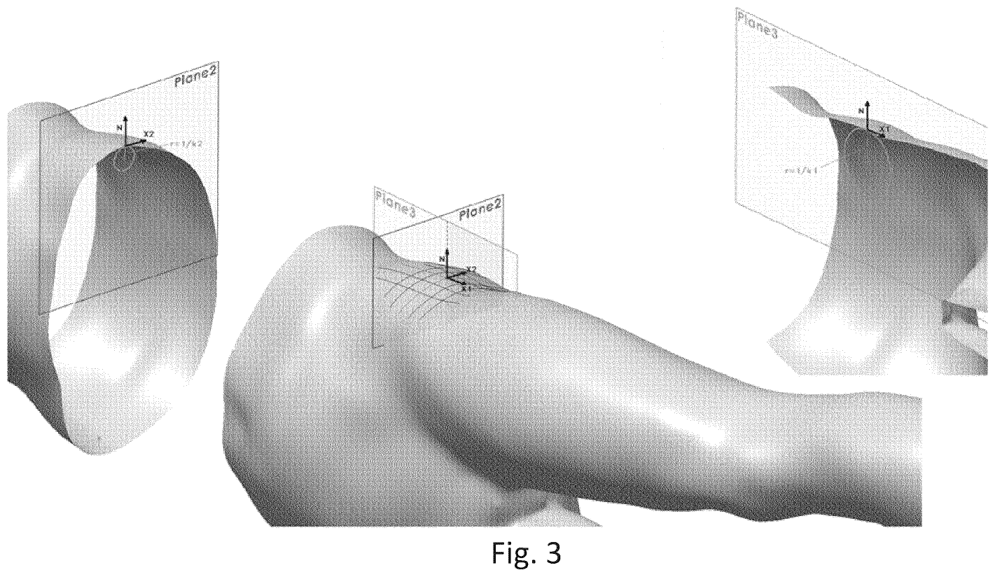

More particularly, with reference to FIGS. 2 and 3, the step b) of automatically deriving anatomical landmarks of parts of the wearer's body from such a three-dimensional digital model is carried out according to the process of extracting anatomical landmarks comprising the following sub-steps:

(i) defining the area of interest on the body surface;

(ii) analysing the local curve in the selected area on that three-dimensional digital model;

(iii) extracting the landmark from the local curve analysis.

The first sub-step (i) for extracting the anatomical landmark is to identify a specific body surface (denoted by δΩ) that contains the anatomical landmark. The extension ranges of these surfaces may be obtained from anatomical tables. As an example of an anatomical landmark, let us consider the left greater trochanter GT_{L} located on a body region δΩ_{GT}_{_{L}} on the left side of the pelvis (FIG. 2, image a)

The next sub-step (ii) for extracting the anatomical landmark is the analysis of the local curvature, or Gaussian curvature k_{g}, for each point belonging to the surface of interest. Let us consider a generic point belonging to the surface, and consider all the planes passing through the normal N to the surface at the point concerned. The intersection of each plane with the surface results in a plane curve whose curvature can be calculated. The maximum and minimum curvature values thus obtained are called main curvatures, and are denoted by k₁ e k₂ respectively. The value of Gaussian curvature for each point belonging to the surface is defined as the product of the two main curvatures, i.e. k_{g}=k₁·k₂. The graphic illustration of the calculation of Gaussian curvature for a generic point on the surface is shown in FIG. 3. By considering the Gaussian curvature of all the points on the surface, it is possible to obtain a colour map representative of the Gaussian curvature for all the points (FIG. 2, image b), where white colour represents higher values of the Gaussian curvature and black colour represents lower values of the Gaussian curvature.

The extraction of the anatomical landmark makes use, for example, of a generic construction from the local curvature analysis. The procedure avails itself of the following construction, illustrated in FIG. 2, image b: firstly, a point Q of construction forming part of the surface δΩ_{GT}_{_{L}} is considered and is joined with a point at the same vertical height z belonging to the axis of the body, thereby drawing a straight line “a”; then a plane W, that includes the point Q and is orthogonal to the straight line “a” drawn in the previous point, is defined; then the two extreme points representing the ends of the zone where there is a maximum Gauss curvature are projected onto this plane and the segment Y is traced on the plane joining the two points; then the segment thus obtained is projected onto the scanning surface, so as to obtain a curve Γ and the line t tangent to such curve is considered; finally, the anatomical landmark GT_{L} is given by the tangent point between Γ and t (FIG. 2, image c).

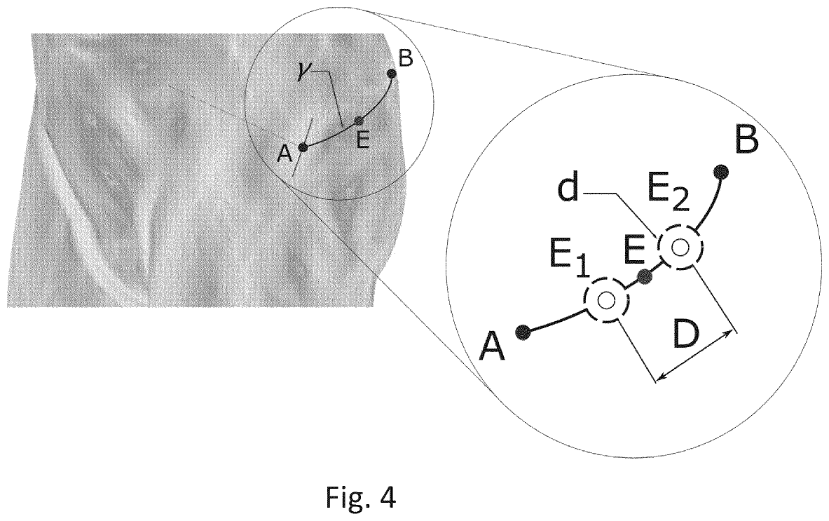

With reference to FIG. 4, the sub-step c) of automatically calculating, from such anatomical landmarks, the positioning coordinates on the three-dimensional digital model previously provided during step a), of at least one electromyography (EMG) electrode device, is graphically shown.

The step c) of automatically calculating the coordinates for positioning, on such three-dimensional digital model, at least one electromyography (EMG) electrode device comprises the sub-step of identifying a curve joining a first anatomical landmark and a second anatomical landmark, wherein the coordinates for positioning said at least one electromyography (EMG) electrode device identify a pair of positioning points belonging to such curve, in particular a first positioning point for such first electromyography (EMG) electrode device and a second positioning point for such second electromyography (EMG) electrode device.

In particular, the sub-step of identifying a curve joining a first anatomical landmark and a second anatomical landmark may comprise the following further sub-steps:
identifying a curve joining the first anatomical landmark A and the second anatomical landmark B, wherein the curve is the shortest curved line joining the first anatomical landmark A and the second anatomical landmark B following the surface of such three-dimensional digital model;
identifying a construction point E on such curve, the construction point E being at a position between the first anatomical landmark A and the second anatomical landmark B;
with reference to FIG. 4, identifying the first positioning point E1 for the first electromyography (EMG) electrode and the second positioning point E2 for the second electromyography (EMG) electrode, within such curve, wherein the first positioning point E1 for the first electromyography (EMG) electrode and the second positioning point E2 for the second electromyography (EMG) electrode are symmetrical, along such curve, with respect to such construction point E.

According to a particular embodiment of the present invention, said sub-step of identifying a construction point on said curve, the position of the construction point E corresponds to the position where the curve has the maximum curvature.

In particular, the position of the construction point E, as will be seen below, is identified at the point deemed to express the maximum potential difference for the muscle to which the first EMG electrode and the second EMG electrode are subsequently applied, and the position of the construction point E limits the cross talk phenomena of adjacent muscles

More particularly, the step c) may be performed as explained in the following paragraphs and with reference to FIG. 4, wherein “E1” and “E2” are the points where the two electrodes are to be positioned, such that the distance on the curve between “E1” and “E” is equal to the distance, also measured on the curve, between “E” and “E2”, “E” being the aforementioned construction point, “d” is the diameter of the textile electrode, equal for the two electrodes (called “first factor” in the subsequent optimisation step), D is the inter-electrode distance (called “second factor” in the subsequent optimisation step).

The coordinates for positioning the electrodes are calculated from the anatomical landmarks and are defined in such a way that they lie on the curved line joining two specific anatomical landmarks A and B at a predetermined distance from the first of the two.

In detail for the positioning of the pair of electrodes, the position Eᵢ is obtained (where i denotes the generic muscle and wherein for each muscle i there is a value Eᵢ).

Hereinafter, the procedure for positioning a textile electrode at the three-dimensional position of the construction point E is illustrated.
Given two anatomical landmarks A and B with coordinates (x_{A}, y_{A}, z_{A}) and (x_{B}, y_{B}, z_{B}),
Given γ the shortest curved line joining A and B following the body surface,
Given l(γ) the length of such curve,.
The construction point E (x_{E}, y_{E}, z_{E}) belongs to γ and is located at a length from A (first anatomical landmark) calculated with the following formula I:

$$l ( A E ) = α * l ( γ ) \qquad ( I )$$

According to the SENIAM guidelines, given the coordinates of the anatomical landmarks related to the muscles of interest (i.e. points A and B) and the percentage value α, it is possible to define point E and thus the positioning of the pairs of electrodes for each single muscle in the muscle sector.

Thus, the length of such curve from such first landmark A to such positioning point E is obtained using formula I:

$$l ( A E ) = α * l ( γ ) \qquad ( I )$$

wherein l(AE) is the length of that curve from such first anatomical landmark to such positioning point, α is a percentage value specific to each muscle, and l(γ) is the overall length of such curve.

In particular, α is a percentage value (between 0 and 100), specific to each muscle, as shown below in Table 1:

TABLE 1

| Muscle monitored (right | |
|---|---|
| and/or left side of the body) | α (in percentage) |
| Gluteus Maximus | 50% |
| Rectus Femoris | 50% |
| Vastus medialis | 80% |
| Vastus lateralis | 66% (⅔) |
| Biceps Femoris | 50% |
| Semitendinosus | 50% |

The values of α are provided by the SENIAM guidelines and are valid for all subjects.

Starting from the construction point E identified for the positioning of the sensors, moving along the line γ, symmetrically in both directions, the points for positioning the two electrodes required for electromyographic detection are obtained, as shown in the enlargement in FIG. 4.

According to the present process, the step e) may be performed as explained in the following paragraphs.

For the dimensioning of the electrode and the inter-electrode distance, the method proposed requires a specific experimental campaign.

In detail, a specially constructed factorial plan is used to define the tests to be performed to obtain information in order to give the utmost robustness to the electric functioning of the product.

The factorial plan is based on two control factors: (i) electrode dimension (denoted as the first factor, in FIG. 4 denoted by “d”); (ii) inter-electrode distance (denoted as the second factor, in FIG. 4 denoted by “D”). These two factors are assigned n different values (i.e. levels).

Two factors that are actually disturbing (i.e. not controllable while wearing the garment) are also controlled during the tests:
muscle variability, meaning the different speed in muscle activation and the different number of activated muscle units between the dominant and non-dominant sides (denoted as the third factor);
the conditions of the skin, with or without sweat during intense physical activity (denoted as the fourth factor).

For each test, the output provided for each muscle is then evaluated, i.e. a value of the potential difference between the first electrode and the second electrode as a function of time. The raw data obtained is appropriately processed through a filtering process (e.g. low-pass filter, high-pass filter) and signal rectification. The Root Mean Square (RMS) is calculated for two steps: (i) burst interval, identified as the interval wherein the signal has an amplitude greater than 30% of the maximum value; (ii) baseline interval defined as the interval wherein the signal has an amplitude less than 5% of the maximum value. Finally, the relative Signal to Noise Ratio (SNR) of the movement is calculated according to formula II below:

$$S N {R}_{i} = \frac{R M {S}_{burst , i}}{R M {S}_{baseline , min}} \qquad ( II )$$

where i is the index indicating the generic muscle i.

The variable that measures the product performance, the output of the experiment, is the signal-to-noise ratio (denoted by SNR) of the electrical signal. For the evaluation of the combination of factors (i.e. electrode dimension and inter-electrode distance) in order to make the performance as insensitive as possible to the effect of the factors, the signal-to-noise (S/N) function is used, which highlights the effects of the factors on the variability of the performance (in our case the SNR value). The higher the ratio, the more robust its performance. In the present case, the nominal S/N function selected is “Higher is Better”, i.e. the highest resulting value is selected, in that the highest possible SNR value is desirable. The “Higher is Better” S/N function for SNR performance is hereinafter reported, according to formula III:

$$S / N = - 10 {log}_{10} ( \frac{1}{n} {\sum }_{i = 1}^{n} \frac{1}{S N {R}_{i}^{2}} ) \qquad ( III )$$

The combination of the design factors at a specific level that maximises the average S/N value allows to define the optimal values of dimension and inter-electrode distance.

Advantageously, as aforementioned said, the process of the present invention allows to minimize cross talk phenomena between adjacent muscles, this being possible in particular thanks to the signal-to-noise analysis.

### Embodiment of a Pair of Shorts Manufactured by the Invention Process

By performing the method according to the present invention, a pair of electromyographic shorts, specifically designed for the game of football, was manufactured.

Six muscles of interest (i.e., gluteus maximus, rectus femoris, vastus medialis, vastus lateralis, biceps femoris and semitendinosus) were selected for the purpose of manufacturing the pair of electromyographic shorts to monitor football movements.

Once performed the steps a) and b) of the process, the coordinates of the respective anatomical landmarks proposed by SENIAM (reported in Table 2, where “first landmark” is point A with reference to FIG. 4 and “second landmark” is point B) were identified from the analysis of the body surfaces.

TABLE 2

| | | FIRST LANDMARK | | | | SECOND LANDMARK | | | |
|---|---|---|---|---|---|---|---|---|---|
| MUSCLE MONITORED | | | x | y | z | | x | y | z |
| Gluteus Maximus | dx | Great Trochanter | −18.37 | −165.86 | −9.52 | Sacral Vertebra | −124.6 | 0.28 | 69.61 |
| | sx | | −28.78 | 164.86 | −36.14 | | −124.6 | −0.28 | 69.61 |
| Rectus femoris | dx | anterior superior | 48.06 | −102.69 | 31.64 | superior part | 8.73 | −126.46 | −416.87 |
| | sx | illac spine | 48.60 | 112.05 | 22.73 | of the patella | −5.63 | 117.58 | −438.38 |
| Vastus medialis | dx | anterior superior | 48.06 | −102.69 | 31.64 | anterior border of | −31.41 | −71.01 | −402.19 |
| | sx | iliac spine | 48.60 | 112.05 | 22.73 | the medial ligament | −48.65 | 64.36 | −421.89 |
| Vastus lateralis | dx | anterior superior | 48.06 | −102.69 | 31.64 | lateral part | 1.10 | −138.62 | −424.6 |
| | sx | iliac spine | 48.60 | 112.05 | 22.73 | of the patella | −17.96 | 146.64 | −437.99 |
| Biceps Femoris | dx | ischial | −110.56 | −57.36 | −115.98 | lateral epicondyle | −66.20 | −172.05 | −399.85 |
| | sx | tuberosity | −106.52 | 33.62 | −123.87 | of the tibia | −84.41 | 175.51 | −434.82 |
| Semitendinosus | dx | ischial | −110.56 | −57.36 | −115.98 | medial epicondyle | −46.34 | −82.26 | −444.81 |
| | sx | tuberosity | −106.52 | 33.62 | −123.87 | of the tibia | −71.12 | 78.09 | −463.18 |

From these, it is then possible to calculate the coordinates for positioning the electromyographic textile electrodes on the curved line joining two anatomical landmarks at a given distance from the first of the two reported in Table 3 in percentage terms.

TABLE 3

| | | | POINT E | | |
|---|---|---|---|---|---|
| MUSCLE MONITORED | | α | x | y | z |
| Gluteus Maximus | dx | 50% | −114.16 | −105.36 | 19.39 |
| | sx | | −125.49 | 102.37 | 3.87 |
| Rectus femoris | dx | 50% | 61.80 | −124.54 | −195.01 |
| | sx | | 53.95 | 123.15 | −207.82 |
| Vastus medialis | dx | 80% | −33.47 | −56.58 | −313.85 |
| | sx | | −19.44 | 54.17 | −332.96 |
| Vastus lateralis | dx | ⅔ | 46.42 | −137.38 | −274.73 |
| | sx | | 28.52 | 150.68 | −284.41 |
| Biceps Femoris | dx | 50% | −118.53 | −116.41 | −259.03 |
| | sx | | −120.33 | 105.23 | −280.81 |
| Semitendinosus | dx | 50% | −94.11 | −55.17 | −280.40 |
| | sx | | −94.31 | 48.02 | −293.76 |

The optimisation step e) is then carried out by detecting the electromyographic signal while performing typical football movements.

In the example proposed, circular surface electrodes of two different diameters were used (first factor, denoted by “d” in FIG. 2): 24 mm (denoted by the “−” sign in Table 4) and 48 mm in diameter (denoted by the “+” sign in Table 4). The factorial plan underlying the experiment provides the test to be repeated for two different electrode distances (second factor, denoted by “D” in FIG. 2), they are then placed at the distances of 48 mm (denoted by the sign “−” in Table 4) and 56 mm (denoted by the sign “+” in Table 4). The two disturbance factors are also controlled during the tests: (i) variability between limbs (third factor), with two levels, dominant limb (denoted by “−” sign in Table 4) and non-dominant limb (denoted by “+” sign in Table 4); (ii) skin lubrication condition (fourth factor) with two levels, dry skin (denoted by “−” sign in Table 4) and sweaty skin (denoted by “+” sign in Table 4).

Measurements were carried out while performing two different athletic movements typical of football (volley and ball control) and on 6 different muscle groups of interest: Gluteus Maximus, Rectus Femoris, Vastus Medialis, Vastus Lateralis, Biceps Femoris, Semitendinosus.

For example, the monitoring of the rectus femoris while performing the volley provides the following SNR values (Table 4):

TABLE 4

| | | − | − | + | + | third factor |
|---|---|---|---|---|---|---|
| | | − | + | − | + | fourth factor |
| − | − | 14217.19 | 11774.95 | 13951.37 | 11701.4 | |
| − | + | 26547.02 | 9933.07 | 12808.08 | 27126.8 | |
| + | − | 15399.22 | 8790.38 | 12439.45 | 12061.7 | |
| + | + | 44190.03 | 25542.64 | 12619.02 | 14319.3 | |
| first | second | | | | | |
| factor | factor | | | | | |

| AVERAGE | STD DEV. | S/N |
|---|---|---|
| 12996.06 | 1726.94 | 82.11 |
| 18240.05 | 11747.84 | 83.23 |
| 12094.80 | 4673.16 | 81.18 |
| 34866.34 | 13185.70 | 84.81 |

The S/N objective function is maximised by the +/+configuration (score underlined in Table 4): 48 mm diameter electrodes placed at an inter-electrode distance of 56 mm.

Below is a summary table of the final configurations for each muscle (Table 5).

TABLE 5

| | First factor | Second factor |
|---|---|---|
| GLUTEUS MAXIMUS | 24 mm | 56 mm |
| RECTUS FEMORIS | 48 mm | 56 mm |
| VASTUS MEDIALIS | 48 mm | 56 mm |
| VASTUS LATERALIS | 48 mm | 56 mm |
| BICEPS FEMORIS | 24 mm | 48 mm |
| SEMITENDINOSUS | 48 mm | 56 mm |

Thus, unlike the prior art, the present invention allows to calculate, for each muscle, different dimensions for diameter and inter-electrode distance, customising the garment for each person.

Furthermore, in the present example, the step d) of making available the above-mentioned pair of shorts was previously performed as follows:
automatically deriving anthropometric measures of the wearer's leg from a three-dimensional digital model;
obtaining the garment dimensions from the anthropometric measures of the wearer's leg, thus derived;
manufacturing a garment having the dimensions thus obtained.

In particular, during the sub-step of obtaining the dimensions of the garment, the garment dimensions are calculated taking into account the elasticity of the material to be used for manufacturing the garment.

In the present case, starting from six anthropometric measures that can be assessed by scanning the athlete (waist circumference, maximum thigh circumference, minimum thigh circumference, hip height, thigh length, crotch width), the following measures are then calculated for manufacturing the shorts: waist tubular body circumference, largest thigh tubular body circumference, smallest thigh tubular body circumference, waist tubular body height, thigh tubular body height, crotch arch length) and taking into account the elasticity of the material it was possible to obtain the exact measures of the shorts (worn and not worn).

From the dimensions obtained from the athlete's scan, taking into account the elasticity of the material (e.g. 79% polyamide and 21% elastane fabric), the product dimensions were obtained.

Assuming that the worn product involves the average elongation allowed by the material, the dimensions reported in Table 6 below are obtained.

TABLE 6

| Mᵢ [mm] | aᵢ [%] | mᵢ [mm] |
|---|---|---|
| M1 = 764 | a₁ = 125 | m₁ = 611 |
| M₂ = 543 | a₂ = 125 | m₂ = 435 |
| M₃ = 405 | a₃ = 125 | m₃ = 324 |
| M₄ = 222 | a₄ = 140 | m₄ = 160 |
| M₅ = 229 | a₅ = 140 | m₅ = 164 |
| M₆ = 52.0 | a₆ = 140 | m₆ = 39 |

Wherein in Table 6, we have Mᵢ=anthropometric measures; αᵢ=average percentage elongation of the material; mᵢ=manufacturing measures of the shorts. M₁=waist circumference; M₂=maximum thigh circumference; M₃=minimum thigh circumference; M₄=hip height; M₅=thigh length; M₆=crotch width; m₁=waist tubular body circumference; m₂=largest thigh tubular body circumference; m₃=smallest thigh tubular body circumference; m₄=waist tubular body height; m₅=thigh tubular body height; m₆=crotch arch length.

According to the present example, the above-mentioned measures were obtained from six anthropometric measures extracted from the three-dimensional digital model and taking into account the elasticity of the material. From a formal point of view, the above-mentioned six measures were obtained using the following formula (IV):

$$m = M / a \qquad ( IV )$$

where “m” is the garment's manufacturing measure; “M” is the corresponding anthropometric measure obtained from the three-dimensional digital model and “a” is the elongation of the material (not in percentages; Example: for an elongation of 125%, we have a=1.25).

FIG. 4 shows a schematic version of the pair of shorts 1 manufactured according to the embodiment just described, comprising six electromyography (EMG) electrode devices 2 i.e. six pairs of electromyography (EMG) electrodes.

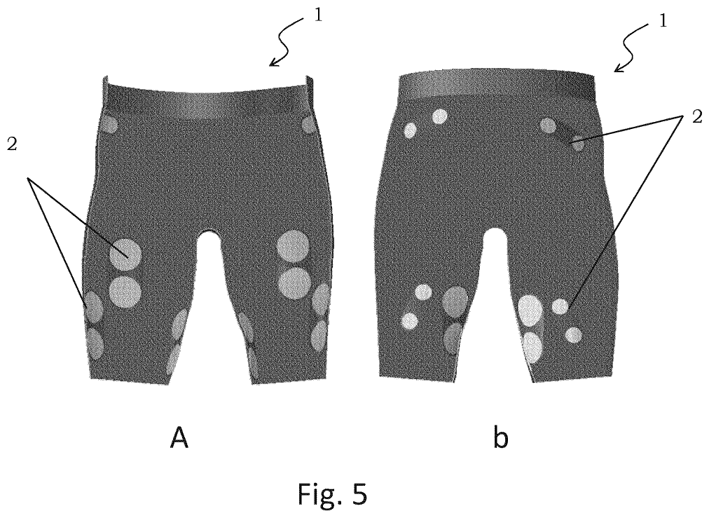

FIG. 5 shows the wiring and electronic components comprised in the same pair of shorts 1 as per FIG. 4.

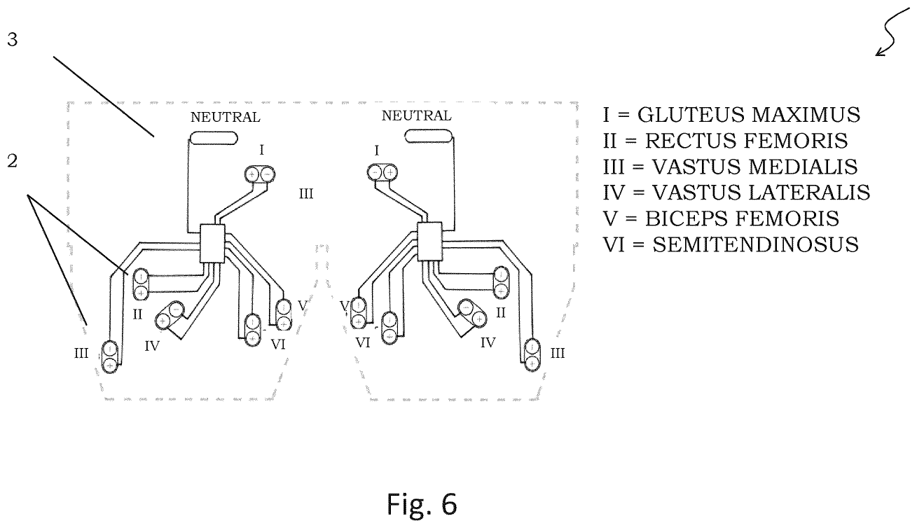

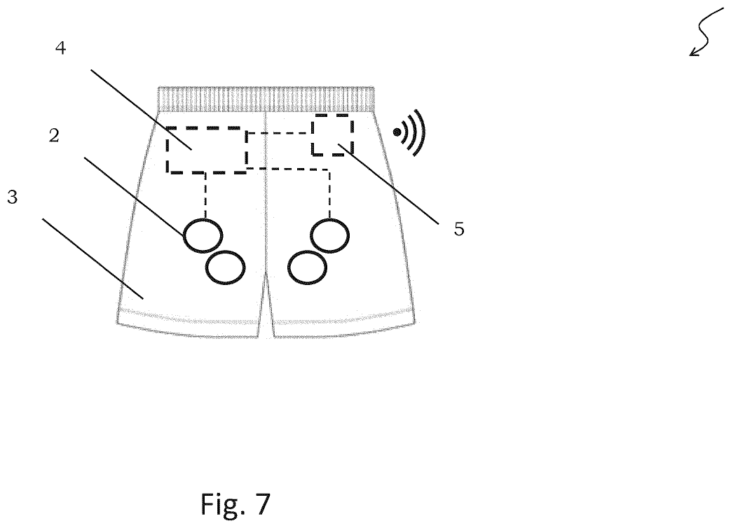

As illustrated, with reference to both FIG. 6 and FIG. 7, wherein a different embodiment of the garment according to the present invention is shown, still in the form of a pair of shorts, the garment 1 according to the present invention comprises a base structure 3 i.e. a mesh or support fabric, useful for anchoring the electronic components, as well as at least one control unit 4, which is also part of the electronic components.

As illustrated, the at least one control unit 4 is connected to the electromyography (EMG) electrode devices 2, being suitably arranged to receive a plurality of signals from such at least one electromyography (EMG) electrode device.

At least one control unit 4 is also arranged to process information associated with such a plurality of signals from electromyography (EMG) electrode devices 2.

The garment 1 comprises a unit for transmitting information 5, optionally in a wireless mode.

1. A process for manufacturing a garment (1) for acquiring electromyographic signals comprising the following steps:
a) providing a three-dimensional digital model of anatomical shapes of a wearer;
b) automatically deriving, from said three-dimensional digital model, anatomical landmarks of body parts of the wearer;
c) automatically calculating, from said anatomical landmarks, positioning coordinates, on said three-dimensional digital model, of at least one electromyography (EMG) electrode device (2), said at least one electromyography (EMG) electrode device (2) comprising a first electromyography (EMG) electrode and a second electromyography (EMG) electrode;
d) providing a garment having dimensions consistent with said three-dimensional digital model of anatomical shapes of a wearer, thus provided during step a), said at least one electromyography (EMG) electrode device (2), preferably a textile electrode device, being positioned on said garment according to said positioning coordinates, said first electromyography (EMG) electrode and said electromyography (EMG) electrode being positioned on said garment in such a way that they both lie along the same muscle;
wherein said step c) of automatically calculating positioning coordinates of at least one electromyography (EMG) electrode device (2) comprises the step of identifying a curve connecting a first anatomical landmark and a second anatomical landmark, wherein the positioning coordinates of said at least one electromyography (EMG) electrode device identify a couple of positioning points belonging to said curve, in particular a first positioning point for said first electromyography (EMG) electrode and a second positioning point for said second electromyography (EMG) electrode, said step of identifying a curve joining a first anatomical landmark and a second anatomical landmark including the following further sub-steps:
identifying a curve joining said first anatomical landmark and said second anatomical landmark, wherein said curve is the shortest curved line joining the first anatomical landmark and the second anatomical landmark following the surface of such three-dimensional digital model;
identifying a construction point on said curve, such construction point being in a position between such first anatomical landmark and such second anatomical landmark;
identifying the first positioning point for the first electromyography (EMG) electrode and the second positioning point for the second electromyography (EMG) electrode, on said curve, wherein the first positioning point for the first electromyography (EMG) electrode and the second positioning point for the second electromyography (EMG) electrode are symmetrical, along that curve, with respect to said construction point; and
wherein said step d) of providing a garment comprises the following sub-steps:
automatically deriving, from said three-dimensional digital model, anthropometric measures of body parts of the wearer;
obtaining, from the anthropometric measures of the body parts of the wearer thus derived, the dimensions of the garment, the dimensions of the garment being calculated taking into account the elasticity of the material to be used for manufacturing the garment;
manufacturing a garment having the dimensions thus obtained, wherein said garment has dimensions consistent with said three-dimensional digital model of anatomical shapes of a wearer, thus made available during step a), said at least one electromyography (EMG) electrode device (2), preferably a textile electrode device, being positioned on said garment according to said positioning coordinates.

2. The manufacturing process according to claim 1, wherein said step a) of providing a three-dimensional digital model comprises the following sub-steps:
scanning anatomical shapes of the wearer, thereby acquiring information related to said anatomical shapes;
reconstructing a three-dimensional digital model of the anatomical shapes of the wearer from the information thus acquired.

3. The manufacturing process according to claim 2, wherein in the sub-step of reconstructing a three-dimensional digital model, said three-dimensional digital model is reconstructed by photogrammetry.

4. The manufacturing process according to anyone of claims 1 to 3, wherein in said sub-step of identifying a construction point on said curve, in said position between such first anatomical landmark and such second anatomical landmark the curve has the maximum curvature.

5. The process according to any one of claims 1 to 4, wherein, after said step d) of providing a garment, the following further steps are carried out:
e) optimising the dimensioning of said first electromyography (EMG) electrode and of said second electromyography (EMG) electrode and/or optimising the distance between said first electromyography (EMG) electrode and said second electromyography (EMG) electrode, by measuring the electric potential difference between said first electromyography (EMG) electrode and said second electromyography (EMG) electrode, thereby obtaining an optimisation outcome;
f) repositioning on the garment said first electromyography (EMG) electrode and said second electromyography (EMG) electrode according to said optimisation outcome.

6. The process according to claim anyone of claims 1 to 5, wherein the optimisation step e) is carried out by measuring the electric potential difference between said first electromyography (EMG) electrode and said second electromyography (EMG) electrode when the wearer performs a specific athletic movement, during which said muscle, on which the first electromyography (EMG) electrode and the second electromyography (EMG) electrode are positioned, is in contracted state.

7. The process according to anyone of claims 1 to 6, wherein the optimisation step e) is carried out by measuring the electric potential difference between said first electromyography (EMG) electrode and said second electromyography (EMG) electrode, varying the distance between said first electromyography (EMG) electrode and said second electromyography (EMG) electrode on the garment.

8. The process according to anyone of claims 1 to 6, wherein the optimisation step e) is carried out by measuring the electric potential difference between said first electromyography (EMG) electrode and said second electromyography (EMG) electrode, varying the dimensions of said first electromyography (EMG) electrode and/or said second electromyography (EMG) electrode on the garment.

9. The process according to anyone of claims 1 to 6, wherein the optimisation step e) is carried out by measuring the electric potential difference between said first electromyography (EMG) electrode and said second electromyography (EMG) electrode, varying the distance between said first electromyography (EMG) electrode and said second electromyography (EMG) electrode on the garment, and
by measuring the electric potential difference between said first electromyography (EMG) electrode and said second electromyography (EMG) electrode, varying the dimensions of said first electromyography (EMG) electrode and/or said second electromyography (EMG) electrode on the garment.

10. The process according to claim 9, wherein the optimisation step e) is carried out by additionally measuring the electric potential difference between said first electromyography (EMG) electrode and said second electromyography (EMG) electrode, varying the position of said first electromyography (EMG) electrode and said second electromyography (EMG) electrode on the garment in terms of positioning with respect to muscles located on the opposite mirror-like side of the body of the wearer.

11. The process according to claim 9, wherein the optimisation step e) is carried out by additionally measuring the electric potential difference between said first electromyography (EMG) electrode and said second electromyography (EMG) electrode by varying the position of said first electromyography (EMG) electrode and said second electromyography (EMG) electrode on the garment in terms of positioning relative to the variation of the sweating conditions of the wearer.

12. The process according to claim 9, wherein the optimisation step e) is carried out by additionally measuring the electric potential difference between said first electromyography (EMG) electrode and said second electromyography (EMG) electrode varying the position of said first electromyography (EMG) electrode and said second electromyography (EMG) electrode on the garment in terms of positioning with respect to muscles located on the opposite mirror-like side of the body of the wearer, and
by additionally measuring the electric potential difference between said first electromyography (EMG) electrode and said second electromyography (EMG) electrode by varying the position of said first electromyography (EMG) electrode and said second electromyography (EMG) electrode on the garment in terms of positioning relative to the variation of the sweating conditions of the wearer.

13. A garment (1) comprising a base structure (3), at least one electromyography (EMG) electrode device (2), preferably said electromyography (EMG) electrode device (2) being a textile electrode device, and a control unit (4), said control unit (4) being connected to said at least one electromyography (EMG) electrode device (2) and is configured to receive a plurality of signals coming from said at least one electromyography (EMG) electrode device (2), obtained by the process according to any one of claims 1 to 12, preferably comprising a unit for transmitting information (5), optionally in a wireless mode.
